(19) United States
(12) Patent Application Publication
(10) Pub. No.: US 2026/0283822 A1
(43) Pub. Date: Sep. 24, 2026

(54) Prosthetic Foot

(71) Applicant: Icarus Medical, LLC, Charlottesville, VA (US)

(72) Inventors: David T. Johnson, Charlottesville, VA (US); Evan Eckersley, Charlottesville, VA (US); Philip Miller, Charlottesville, VA (US); George Miroulis, Virginia Beach, VA (US)

(21) Appl. No.: 19/572,731

(22) Filed: Mar. 19, 2026

Related U.S. Application Data
(60) Provisional application No. 63/774,734, filed on Mar. 19, 2025.

(51) Int. Cl. A61F 2/66; A61F 2/50

(52) U.S. Cl. CPC A61F 2/6607; A61F 2002/503; A61F 2002/6642; A61F 2002/665

(57) ABSTRACT

An assistive foot orthosis.

### CROSS-REFERENCE TO RELATED APPLICATION

The present application relies on the disclosures of and claims priority to and the benefit of the filing date of U.S. patent application Ser. No. 63/774,734, filed Mar. 19, 2025. The disclosures of that application are hereby incorporated by reference herein in their entireties.

### BACKGROUND

### Field of the Invention

Prosthetic foot with a user adjustable energy absorption or storage mechanism.

### Description of Art

In the United States, approximately 150,000 people undergo a lower extremity amputation every year typically due to diabetes or trauma. It has been estimated that two thirds of lower extremity amputations are below the knee (BK). A large percentage of BK amputees are candidates for prosthetic devices.

Prosthetic feet are essential devices for individuals with lower-limb amputations, enabling them to regain mobility and improve quality of life. However, despite advances in materials and design, several significant challenges remain which limit the effectiveness of current prosthetic feet. The key issues include lack of adaptability, inadequate replication of human biomechanics, and high costs, among others.

One of the primary problems with current prosthetic feet is their limited adaptability to various terrains and walking conditions. While the human foot can dynamically adjust to different surfaces (e.g., inclines, uneven ground), most prosthetic feet offer limited or no dynamic adaptation. Most available prosthetics are either passive (unable to respond in real-time to changing conditions) or semi-active, meaning users must rely on compensatory movements to adjust, which can lead to long-term strain and discomfort.

Replicating the complex movements of the human foot is a significant challenge. The foot and ankle comprise 26 bones. The subtalar joint contributes to supination and pronation motions of the foot while the distal ends of the metatarsal bones help with weight bearing. The first and fifth metatarsal bones have a large range of motion up and down which allows the foot to adapt to uneven terrain. The two sesamoid bones lie under the first metatarsal and help redistribute forces during toe-off. The bones of the foot are inter-connected by ligaments and muscles such that none of the bones between the rear of the calcaneus and the heads of the metatarsals in the front touch the ground. The arch between the calcaneus and the metatarsals is locked and unlocked during normal walking gait by forces applied by the muscles and ligaments of the foot and ankle. During heel strike the arch is unlocked providing a large amount of flexibility to accommodate uneven terrain. During toe-off the arch is locked creating a large lever arm to propel the person forward.

In addition to the biodynamics described above, the natural foot provides feedback and shock absorption during walking. Many prosthetic designs struggle to fully mimic these biomechanical functions. This can result in inefficient energy use, causing fatigue or discomfort during extended use. Additionally, the lack of proprioceptive feedback (awareness of foot positioning) in most prosthetic feet forces users to rely on visual cues, which are not as effective.

The mismatch between prosthetic foot mechanics and natural walking often places added stress on the rest of the body. Users commonly report issues like hip, knee, and back pain due to unnatural gait patterns and improper weight distribution. Over time, these issues can contribute to degenerative joint problems, significantly impacting the user's health.

The cost of advanced prosthetic feet remains prohibitively high for many. Devices that offer better biomechanical mimicry or energy storage (like microprocessor-controlled feet or carbon fiber prosthetics) are often priced at several thousand dollars, making them inaccessible to a large portion of the population in need. Additionally, maintenance and repairs can add to the long-term costs, further limiting access to these technologies.

For BK amputees, the transfer of force from a prosthetic foot into the body is a significant issue, particularly concerning the health of the knee joint. The lack of proper energy absorption and redistribution in current prosthetic designs causes biomechanical imbalances, often leading to secondary problems in the knees and other joints.

In a natural limb, the foot plays a critical role in absorbing and dissipating ground reaction forces during walking. The foot and ankle joints, along with soft tissues, act as shock absorbers, protecting the knee and other joints from excessive strain. However, many prosthetic feet lack this dynamic energy absorption capability, leading to improper force transfer up the leg.

In a below-knee amputee, the residual limb is often subjected to increased forces because the prosthetic does not effectively absorb shock. This additional force is transferred directly to the knee, causing higher stress on the knee joint. Over time, this can result in knee pain, osteoarthritis, or even degenerative joint diseases due to the repetitive strain on the knee's cartilage and ligaments.

Studies have shown that individuals using prosthetic feet often exhibit higher forces on the knee, especially during walking. The lack of energy return from the prosthetic foot during push-off requires the user to exert more effort, leading to increased stress on the knee during the stance phase of walking. This creates an asymmetry in gait mechanics, which can result in compensatory movements that place additional strain on the knee.

Research suggests that prosthetic users experience higher knee joint moments compared to individuals with intact limbs. Knee joint moments refer to the rotational forces applied to the knee joint during movement, and higher moments can lead to excessive wear and tear over time. BK amputees often must compensate for the lack of propulsion from the prosthetic foot, resulting in higher knee extension moments. This can lead to overuse injuries in the knee joint, further exacerbating long-term knee health issues.

Another issue is the lack of proprioception (the body's ability to sense its position in space) in most prosthetic feet. This forces users to rely on visual cues or adjust their walking mechanics to avoid tripping or stumbling. The result is often unnatural gait patterns, which lead to knee problems due to compensatory movements. For example, the knee may hyperextend or overcompensate during the swing phase, leading to patellofemoral pain syndrome or other joint issues.

Over time, the combination of improper force absorption, higher knee moments, and compensatory gait patterns can lead to osteoarthritis and other degenerative knee conditions. BK amputees using prosthetic feet with poor energy storage and return systems are particularly at risk, as their knee must work harder to stabilize and propel the body forward during walking, leading to cumulative damage.

Thus, there is a need for a prosthetic foot which can mimic natural foot dynamics, easy and inexpensive to make, lightweight, and readily adaptable to a variety of terrain and activities. A prosthetic foot that can be customized to the user's specific anatomy, condition, activity and need and designed for a high degree of energy efficiency would offer benefits over existing technologies.

Some traditional foot prosthetics are substantially inactive. That is, there are no (or limited) moving pieces. Their purpose is to bear the weight of the user securely on the ground without slipping, fit in a shoe, and not to adversely affect the swing of the amputated leg.

Other foot prosthetics, such as the carbon fiber blade foot, are reactive: they are constructed to be resilient and compress during heel strike/weight loading and rebound during toe-off. These are often favored by runners.

A third type of foot prosthetic is active-externally powered actuators driven by a microprocessor are used to mimic foot function during the different stages of the walking gait. The present invention falls somewhere between pure reactive and pure active.

### SUMMARY

An improved prosthetic foot with a user adjustable energy absorption or storage mechanism is described. In one embodiment, impact energy generated during heel strike is absorbed and stored for release upon toe-off to mimic the natural energy loading of the arch of the human foot while walking. The amount of energy absorbed and transferred is readily adjusted by the wearer so that the user can fine tune the action of the novel prosthetic foot to the demands of various activities (running, walking, climbing, etc.) or terrain. The device can be manufactured from high-strength, lightweight materials including additive manufacturing methods in order to improve gait dynamics and provide more personalized form and function compared to existing prosthetic feet.

Embodiments of the present invention include but are not limited to an ankle-foot joint device and system (e.g., prosthetic foot) wherein a forefoot plate component comprises, in aspects, at least one lever that articulates about one or more axes, and a heel plate component that comprises at least one lever that articulates about one or more axes, and each of the levers attached to a tensioning system on one end of the lever, and the other end of the lever makes contact with the ground, directly or indirectly, during use by the wearer. The tensioning system can comprises a cable or lace, an optional tensioning element, such as a spring or elastic member, and a tension adjustment mechanism, such as a ratcheting dial or motor. When the ratcheting dial is turned, the tension in the cables or lace can increase, increasing the downward force to either or both of the forefoot or heel plate components. A shank representing the tibia can be attached to the forefoot and heel plate components, and can house the tensioning element(s), cables/lace, and tension adjuster mechanism. The effect of the devices, in cases, is that when the wearer strikes the ground with the heel plate component (the heel), then the lever pulls on the tensioning system, thereby absorbing shock, and possibly canting the tibial shank forward during gait. Then the forefoot section lever is tensioned and absorbs shock through stance phase and also provides a push-off force until the leg is lifted and the user returns to swing phase.

Aspects of the invention include but are not limited to: a tensioning or compression adjustment mechanism or dial for adjusting force (such as when the user is wearing the prosthesis) (the dial may control just one or a multitude of levers); a tensioning system, which may have one or a multitude of tensioning elements (which may include compression elements); a forefoot plate component that can possess a multitude of levers and tensioning elements; motors, actuators, or sensors; ability to mimic anatomic features; ability to enhance performance, safety, anatomical features, and gait; technology can be applied to a knee (e.g., quadricep and/or hamstring) or hip glutes/hip flexors; the forefoot can have subsystems (such as tarsals/metatarsals/phalanges) that can also be tensioned and can have different tension levels; ability to be built as an orthosis, wherein the articulating and tensioning systems are on the side or sides of the limb; a shock absorbing component could exist in-line with the joint (e.g., ankle); and/or the joint can optionally move and be tensioned across one or more planes to control and adjust pitch, yaw, and roll.

Accordingly, embodiments can include an ankle-foot joint system, comprising a forefoot plate component, which can include at least one lever that articulates about one or more axes; a heel plate component, which can include at least one lever that articulates about one or more axes; a tensioning system attached to one end of each lever in the forefoot and heel plate components, the other end of each lever configured to make contact with the ground, directly or indirectly, during use by a wearer; wherein the tensioning system further comprises a cable or lace, an optional tensioning element, and/or a tension adjustment mechanism; wherein adjusting the tensioning system changes tension (and/or compression) in the cable or lace, thereby changing the downward force on either or both of the forefoot and heel plate components; a tibial shank, connected to both the forefoot and heel plate components, and optionally housing the tensioning elements, cables or lace, and optionally the tension adjustment mechanism; wherein the heel plate component lever, upon striking the ground during gait, pulls on the tensioning system and absorbs shock; and wherein the forefoot plate component lever absorbs shock through the stance phase and provides push-off force at the end of the stance phase. Another version can have a single lever that can be tension-controlled, including with the counterpart for compression.

### BRIEF DESCRIPTION OF THE DRAWINGS

The accompanying drawings illustrate certain aspects of embodiments of the present invention and should not be used to limit the invention. Together with the written description the drawings explain certain principles of the invention.

FIG. 1 is an illustration of the human foot.

FIG. 2 is an illustration of planes and rotations of the human foot.

FIG. 3 is a diagram of the walking gait cycle.

FIG. 4a and FIG. 4b show the axis of rotation of the human foot.

FIG. 5 is a side view of one embodiment of a foot prosthetic according to the present invention.

FIG. 6a and FIG. 6b are orthogonal views of an energy absorbing foot prosthetic according to an embodiment of the present invention.

FIG. 7a and FIG. 7b are orthogonal views of another aspect of a foot prosthetic according to an embodiment of the present invention.

FIG. 8 is an orthogonal view of yet another aspect of a foot prosthetic according to an embodiment of the present invention.

FIG. 9a and FIG. 9b are orthogonal views of an energy storage capable foot prosthetic according to an embodiment of the present invention.

FIG. 10 is an orthogonal view of an energy storage sub assembly according to an embodiment of the present invention.

FIG. 11a and FIG. 11b are illustrations of another embodiment of an energy absorbing foot prosthetic according to an embodiment of the present invention.

### DETAILED DESCRIPTION

What is disclosed are embodiments of novel ankle-foot joint systems.

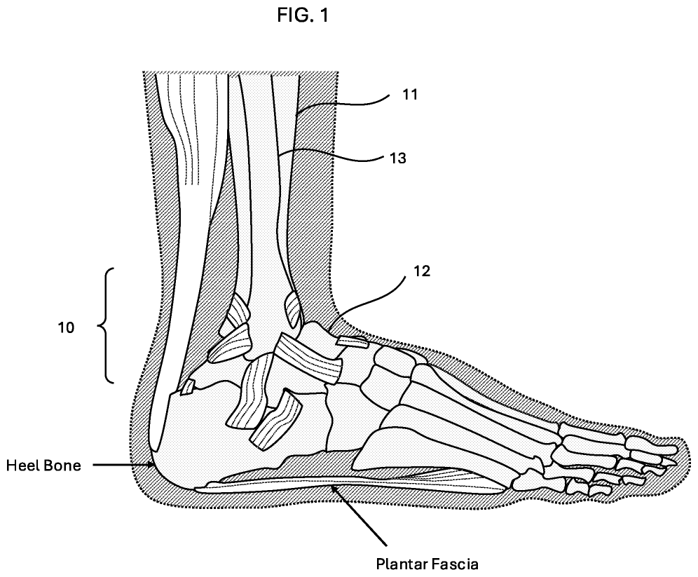

FIG. 1 is an illustration of the major bones, ligaments, and muscles of the foot and ankle. The ankle joint (10) is the connection between the tibia (11) and the talus (12). The fibula (13) is connected to the side of the talus and forms the prominent bump on the lateral side of the ankle. The bump on the medial side of the ankle is from the head of the tibia. The axis of rotation of the foot normal is substantially normal to the sagittal plane and is located slightly above these bumps and the medial side of the axis is slightly forward of the lateral side.

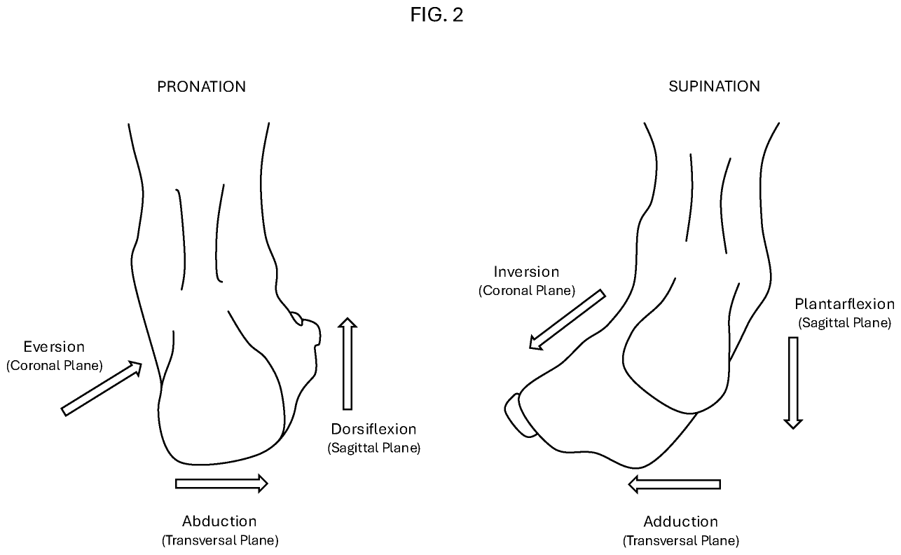

FIG. 2 is an illustration of the rotations of the foot about the planes of the human body. Dorsiflexion and plantarflexion are the upwards and downwards rotations, respectively, of the foot along an axis normal to the sagittal plane. Eversion is the upwards rotation of the lateral side of the foot along an axis normal to the frontal plane whereas inversion is the upwards rotation of the medial side of the foot. Finally, abduction is the rotation of the heel laterally along an axis normal to the horizontal plane and adduction is the rotation of the heel medially. The combination of dorsiflexion, eversion, and abduction is called supination. The combination of plantarflexion, inversion, and adduction is called pronation.

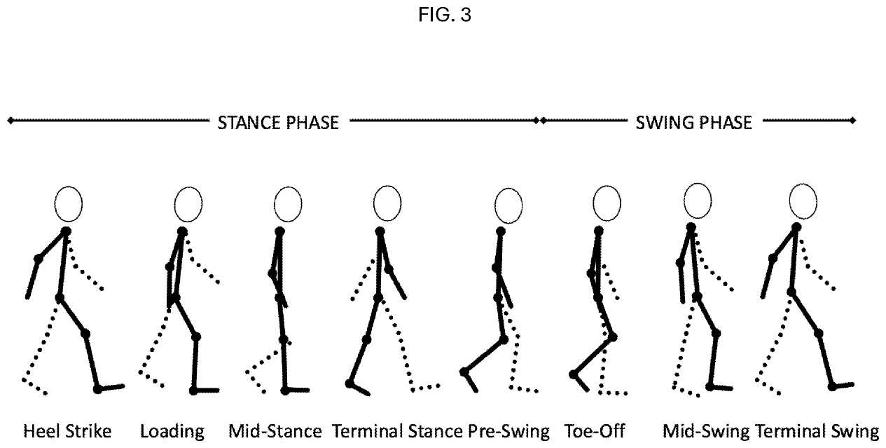

FIG. 3 is an illustration of the different stages of the gait cycle. The start of the walking cycle is at heel strike when the weight of the body first contacts the ground and the body decelerates downwards. As the gait continues the foot rolls forward to the loading response stage of the cycle. At this point the foot is flat against the ground and the walker begins transferring weight from the back of the foot to the forefoot. Next the heel leaves the ground and the forefoot pushes against the ground to propel the walker forwards and upwards. During the swing phase all weight is off the foot as it accelerates forward to swing out in front of the walker to prepare for the next heel strike. The highest ground reaction forces occur just after heel strike and at toe-off. Depending on the pace and terrain the ground reaction forces can be 1-3× the body weight of the walker.

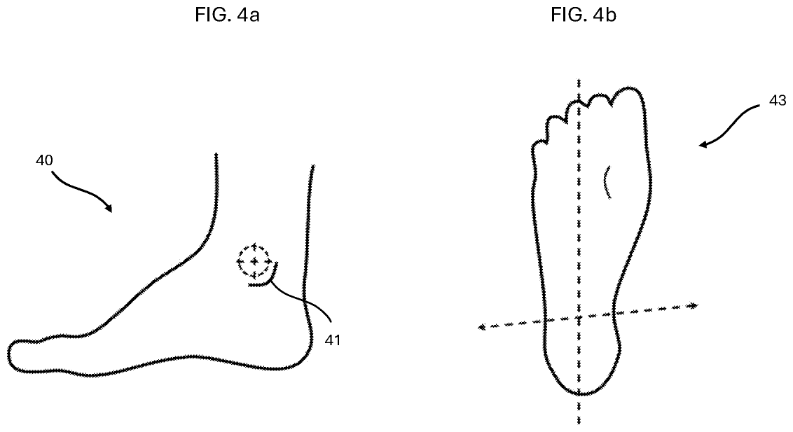

FIG. 4a is a side view of the foot (40). An axis of rotation approximately normal to the sagittal plane is shown as a dashed circle with a cross, slightly above the ankle bone (41). FIG. 4b is a bottom view of the sole of same foot (42). The axis of rotation is shown as the dashed double headed arrow. The foot is divided by a dashed line roughly two thirds laterally from the medial edge of the foot.

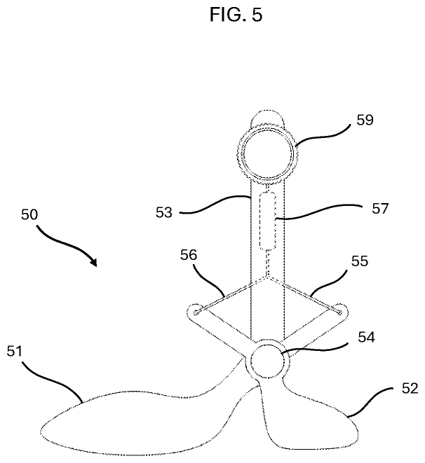

FIG. 5 shows one aspect (50) of the present invention. A forefoot plate component (51) and a heel plate component (52) are attached to a pylon (53) at a pivot point (54). The forefoot plate component and heel plate component can rotate independently of each other. A first flexible tensioning component (55) is connected to a lever arm integrated with the forefoot plate component posterior of the pivot point. A second flexible tensioning component (56) is connected to a lever arm integrated with the heel plate component anterior to the pivot point. The two flexible tensioning components (55) and (56) are connected to a first end of an energy storage component (57). A second end of the energy storage component is connected to an adjustable tensioning component (59) which is attached to the pylon.

A flexible tensioning component may be made from any material or materials that are substantially inextensible and yet flexible and can transmit a tensile force such as a cable, braid, string, chain, lace, strap, rope, combinations therein, or the like. An adjustable tensioning component may be any device that allows the user to increase or decrease a tensile force such as a lever, wheel/spool, screw, ramp, combinations therein or the like. An energy storage component may be any material or device that can transform work from one energy state to a second energy state and later restore substantially most of the work by returning to the first energy state such as an elastic material, a spring, a hydraulic or pressure cell, a flywheel, a battery, combinations therein or the like. An energy storage and transfer mechanism is a mechanism that comprises a flexible tensioning component, an adjustable tensioning component, and an energy storage component.

A non-limiting example of an energy storage and transfer mechanism suitable for the present invention is a thermoplastic polyurethane strip wherein a first end is anchored to the end of the forefoot plate component lever and the second end is tied to a first end of a braided cable of ultra high molecular weight polyethylene fiber. The second end of the braided cable is attached to the spool of a rotary dial with an internal ratchet and pawl mechanism to prevent the spool from turning backwards wherein the rotary dial is attached to the pylon. As the forefoot plate component pivots about the pivot axis in dorsiflexion, by say 15 degrees, the length of the path between the anchored end of the TPU strip and the rotary dial increases which is accommodated by the stretching of the TPU strip. The energy stored in the TPU strip by stretching it can be recovered by allowing the forefoot plate component to rotate in plantarflexion about the pivot plate component back to zero degrees. The amount of energy stored (and subsequently recovered) in the TPU strip resulting from 15 degrees of dorsiflexion can be changed by turning the rotary dial to tighten the braided cable and prestretch on the TPU strip.

As shown in FIG. 5, using the adjustable tension component (59) to increase the pre-tension in the energy storage element (57) affects both the forefoot plate component and the heel footplate component substantially the same due to the symmetry as illustrated. The adjustable tension component may control the position or stored energy within the forefoot, heel, or ankle plate component of the prosthetic foot. Changing the length of the lever arm of the forefoot plate component posterior to the pivot point or the length of the lever arm of the heel plate component anterior of the pivot point would change the ratio of the force transferred by the energy storage element to the forefoot plate component versus the heel plate component. In this manner, it is possible to transfer the energy stored during a long duration/deep heel strike into a short duration/quick rebound forefoot toe-off.

While walking or running it is advantageous to recover the ground reaction force that occurs during deceleration at heel strike and transform it into forward propulsion at toe-off. This mimics the natural action of the foot arch and helps improve the efficiency of walking/running. There are times, however, when it would be advantageous to dissipate the ground reaction force at heel strike rather than store it for a later phase in the walking gait. One such time is coming to a stop after a sprint but another more common occurrence is when descending stairs.

In another aspect of the present invention, a switch or lever allows the wearer to alter the application of force resulting from heel strike to either an energy storage component (such as shown in FIG. 5) or to an energy dissipation component. An energy dissipation component is a material or device where mechanical energy is purposely lost (as a non-limiting example, the energy is transformed to heat via friction). The classic example of an energy dissipation component is a dashpot.

In aspects, the energy storage component and the energy dissipation component can be the same mechanism but depending on the time scale they can act predominantly as one or the other. For example, a piston in a cylinder with no leaks acts as an energy storage component. Push the piston down to compress the gas in the cylinder. Hold the piston in place with a catch to store the energy. Release the catch at a later time and the compressed gas will push the piston up to the original position. If there is a small leak in the cylinder the mechanism will still act as an energy storage component-if the time between pushing the piston down and releasing the catch are short. However, if there is a long delay before releasing the catch, the compressed gas will be lost and the energy will be dissipated instead of stored. Thus, the same mechanism can act as either an energy storage component (short delay) or an energy dissipation component (long delay).

In a similar manner a flywheel could be used as an energy storage component if frictional losses are low or as an energy dissipation component if frictional losses are high. Changing the position or pressure of an element that presses on the flywheel would be a means to allow the user to switch between energy storage and energy dissipation modes of the same mechanism.

The time span between heel strike and toe-off could be employed to automatically alter the mode of the energy storage component into an energy dissipation component. A typical walking pace is 18 minutes per mile with about 2000 steps taken. A step at walking pace takes about 0.5 seconds long. Heel strike to toe-off is about two thirds of the walking gate, or 0.33 seconds. An energy storage component that retains and can return most of the stored energy for at least one third of a second or more will act primarily as an energy storage component for a person walking at an average pace. If >50% of the stored energy is lost after, for example 2 seconds, then the energy storage component would act primarily as an energy dissipation component for a person who stops at a cross-walk and pauses to look both ways before stepping off a curb. In a similar manner, the same energy storage component could shift from energy storage mode during walking to energy dissipation mode while descending stairs at a slower pace.

FIG. 5 shows both flexible tensioning components from the heel plate component lever and the forefoot plate component lever connecting to the same energy storage component (57). In aspects, two energy storage components could be used; a first energy storage element connected to the heel plate component and a second energy storage element connected to the forefoot plate component. The energy storage components could be similar in their ability to store and transfer energy, or they could be different such that, for example, more energy is absorbed/transferred from the heel plate component than the forefoot plate component.

The tensioning adjustment component (59) shown in FIG. 5 is attached to the pylon above the pivot point. In aspects, it is desirable to have a compact prosthetic foot with a low center of gravity. Mounting the tension adjustment component lower on the pylon or below the pivot point is desirable in aspects. In a similar manner, routing the flexible tension component and/or the elastic storage component in or along the foot plate components would make the prosthetic foot of the present inventions more compact with a lower center of gravity without negatively impacting the function. In aspects, the tension adjustment component may be located on a prosthetic or orthotic device or apparel of the wearer (e.g., a belt) and positioned for easy access by the user while still being configured to control the flexible tensioning and energy storage components of the prosthetic foot.

The adjustable tensioning component can be used to add a pre-tension to the energy storage component that can be used to alter the gait mechanics of the wearer. As a non-limiting example, by applying a pre-tensioning to an amount greater than 5× the body weight of the wearer, no ground reaction force will be absorbed by the energy storage component while walking (where typical ground reaction forces are less than 3× the body weight.) In this manner, the prosthetic foot of the present invention could be made to mimic a conventional non-deformable prosthetic foot.

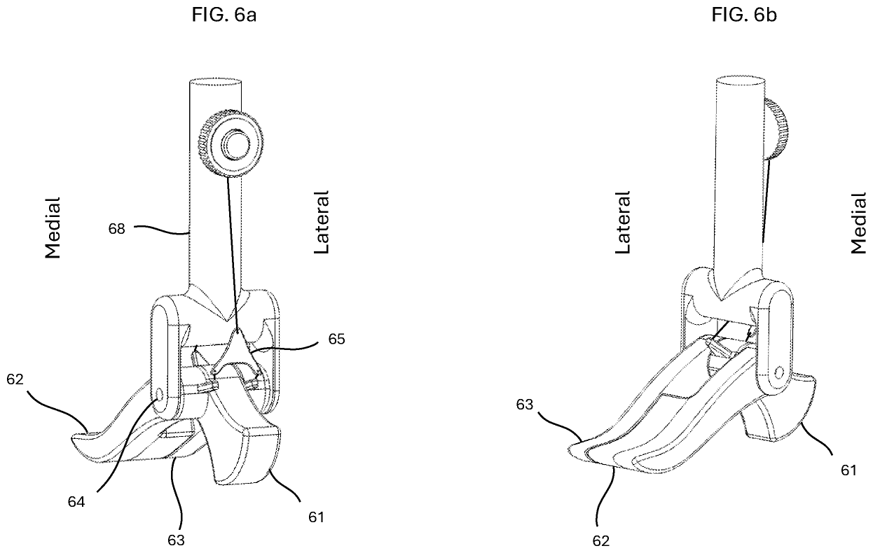

FIG. 6a shows a back orthogonal view of one aspect of the present invention comprised of 3 (three) foot plate component components. The first foot plate component (61) corresponds to the heel of the foot. The second foot plate component (62) corresponds to the medial side of the forefoot. The third foot plate component (63) corresponds to the lateral side of the forefoot. The three components are joined to a pylon component (68) along a common pivot (64) axis. In aspects the size of the medial and lateral forefoot plate components are roughly the same (i.e., split down the middle). In other aspects, as shown in FIG. 6a, one forefoot plate component is larger than the other (e.g., the medial forefoot plate is two thirds the width of the lateral forefoot component). FIG. 6b is a front orthogonal view of the embodiment shown in FIG. 6a.

As shown in FIG. 6a and FIG. 6b, the three foot plate components are not rigidly connected to each other. The two forefoot components (62, 63) are connected to the same end of the energy storage component (65) and substantially rotate about the pivot axis in tandem.

Although the connection points are at the same end of the energy storage component, they are separated laterally by a distance. This lateral separation results in some independence between the degree of rotation of the medial foot plate component (62) and the lateral foot plate component (63). This difference in rotation allows the present invention to mimic supination and pronation of the human foot. Changing the lateral distance or the geometry of the energy storage component at the two connection points will alter the amount of the difference in rotation between the medial foot plate component and the lateral foot plate component and makes the prosthetic foot more stable on uneven terrain.

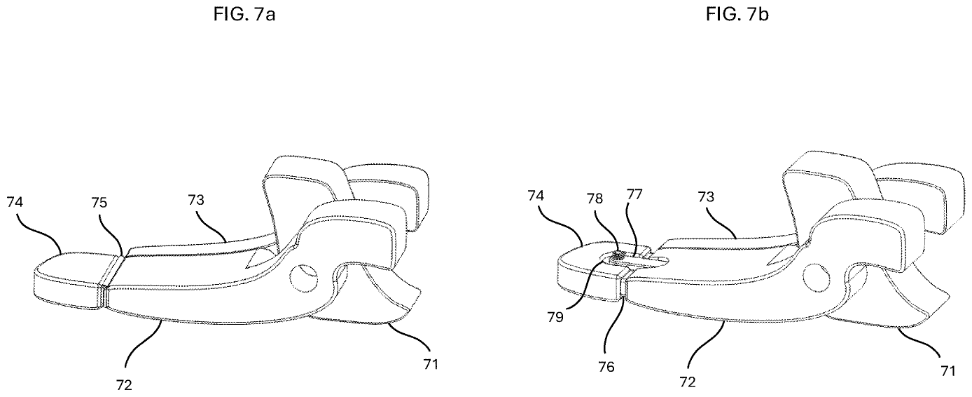

FIG. 7a shows another aspect of a 3 (three) foot plate component prosthetic foot comprising a heel plate component (71), a medial forefoot plate component (72), and a lateral forefoot plate component (73). (The energy storage components can be substantially the same as shown in FIG. 5a or FIG. 6a are not shown in this figure for clarity.) The wherein the medial forefoot plate component (72) has been further divided to generate a toe plate component (74).

The division is located approximately at the connection of the phalanges to the metatarsals in the human foot. A hinge (75) connects the anterior portion to the posterior portion. The hinge (75) is configured to allow the anterior portion to rotate predominantly upwards from an at rest position but not downwards. Suitable hinge components could be, but not limited to, a piano hinge, a flexible elastomer such as a TPU, and the like. The geometry and material properties selected for an elastomeric hinge provide a pre-set amount of torque needed to rotate the toe portion with respect to the rest of the medial foot plate component.

FIG. 7b is an aspect of the present invention wherein the resistance to flexion of the toe plate component portion of the medial forefoot plate component can be changed by the user by, for example, adjusting a set screw (78). As illustrated in FIG. 7b, the toe plate component is connected to the medial foot plate component by an elastomeric block (76). A stiff tongue or beam (77) is connected to the medial foot plate component, and a set screw (78) is threaded into a hole in the tongue or beam. The bottom of the set screw (not shown) protrudes from the tongue or beam and contacts the bottom of a corresponding channel (79) in the toe plate component.

Threading the set screw farther into the tongue or beam causes it to contact the bottom of the toe plate component channel sooner as the toe plate component flexes upwards. This allows the user to change the angle when the toe plate component cannot rotate upwards anymore.

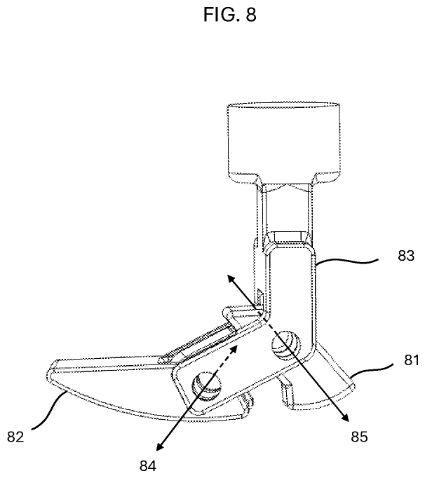

FIG. 8 is an illustration of another aspect of the present invention comprising a pylon (83), a heel foot plate component (81), and a forefoot plate component (82), where in the heel plate component (81) pivots about a first axis (84) and the forefoot plate component (82)(s) pivots about a second axis (85). (The energy storage components can be substantially the same as shown in FIG. 5a or FIG. 6a are not shown in this figure for clarity.) During walking and running, the human foot pivots at the ankle joint and also flexes at the arch and bends at the joints of the phalanges to the metatarsals. The combination of the natural processes of the foot is more like a rolling motion than a pure pivoting motion. By using two pivoting axes (84) and (85), the invention depicted in FIG. 8 can represent the rolling action of the human foot better. In addition, the heel plate component axis (84) need not be parallel to the forefoot plate component(s) axis (85). As shown in FIG. 8, the non-co-axiality of axis (84) and axis (85) work to mimic the natural pronation to supination motion of the human foot from heel strike to toe-off.

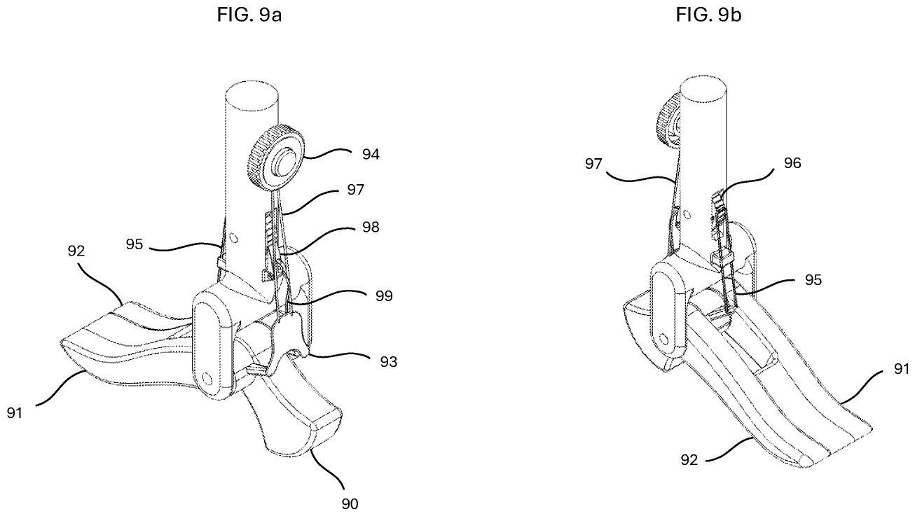

FIG. 9a and FIG. 9b illustrate another embodiment of the current invention. FIG. 9a is a back orthogonal view of the current invention comprising a heel plate component (90), a medial forefoot plate component (91), and a lateral forefoot plate component (92) that are rotatably attached to a pylon. An elastic storage element (93) is connected to the forefoot plate components (91) and (92) and an adjustable tensioning component (94) by a first flexible tensioning component (97). Turning the adjustable tensioning component in a first direction tightens the first flexible tensioning component (97) thereby preloading the elastic storage element (93); turning the adjustable tensioning component in a second direction unloads the elastic storage element. During the phases of the walking stance at which weight is applied to the forefoot plate components (91) and (92), energy is stored in the elastic storage element (93) which is returned as a rebound force to the wearer during the toe-off stage.

During the heel strike stage, the heel plate component (90) pulls a U-shaped wire (95) downwards which causes a wheel (96) to rotate in a first direction. A first end of a torsion spring (not shown) connected to the wheel is loaded as the wheel rotates in the first direction. A pawl (98) prevents the wheel from rotating in a second direction. The U-shaped wire, wheel, torsion spring, and pawl assembly work together to absorb and store the energy generated by heel strike by winding up the torsion spring. The second end of the torsion spring is connected to the first flexible tensioning component (97) by a one-way clutch (not shown). Winding the torsion spring causes the one-way clutch to slide along the flexible tensioning component (97).

The pawl (98) is connected to the forefoot plate component (or components) by a length of a second flexible tensioning component (99). As shown, the length of the second flexible tensioning component (99) is slightly longer than the direct distance between the pawl and the forefoot plate component (or components). As the forefoot plate component (or components) rotate, for example, during the toe-off stage, the distance between the forefoot plate component connection point and the pawl increases. At a predetermined amount of rotation, the distance between the forefoot plate component (or components) and the pawl becomes greater than the length of the second flexible tensioning component (99) pulling on the pawl which releases from the wheel and the torsion spring attempts to return to its initial unloaded condition. The one-way clutch prevents the torsion spring from unwinding thereby transferring the energy stored in the torsion spring to the first flexible tensioning component (97). In this manner, energy is absorbed during heel strike and temporarily stored until the toe-off stage where it is released and used to increase the toe-off rebound force.

In aspects, the length of the second flexible tensioning component (99) can be adjusted by the wearer, for instance by such non-limiting examples as a worm screw, ratcheting dial, slide, lever, or the like, to alter the degree of rotation of the forefoot plate component(s) before the energy stored in the torsion spring is released. In aspects, the trigger to release the energy stored in the torsion spring could alternately be a force plate on the bottom of the forefoot plate component(s) to release when a certain amount of weight (force) is applied to the forefoot plate component(s). In other aspects, an accelerometer, a position sensor, a rotation sensor, and the like can be used to energize an actuator that releases the torsion spring at a predetermined condition (e.g., weight is removed from the heel plate component, the forefoot plate component achieves 14 degrees of dorsiflexion, etc.). In aspects, the torsion spring can be replaced with other energy storage mechanisms such as, but not limited to, a flywheel, a compression spring, a tension spring, an elastomeric element, opposing magnets, and the like.

During the heel strike stage, the heel plate component touches the ground and as the wearer applies weight to the prosthetic the heel plate component rotates about the common pivot axis which imparts a force to the second elastic storage element. A clutch mechanism prevents the elastic storage element from releasing the stored energy back into the heel plate component. As the wearer continues with their gait, their weight on the prosthetic foot shifts forward and the two forefoot plate components come in contact with the ground. As the forefoot plate components rotate about the pivot axis, a release transfers the energy stored by the second elastic storage element to the first elastic storage element which, in turn, applies torque to the two forefoot plate components. During the toe-off stage, the energy is transferred back to the wearer by springing the two forefoot plate components off the ground. During the transition phase when the prosthetic foot isn't touching the ground the two elastic energy components are able to relax to a non-tensioned state ready to restart the cycle.

In another aspect of the current invention, the heel plate component is connected to the pylon by a rack and pinion mechanism and a slide. A weak spring pulls (or pushes) the heel plate component into its neutral position (neither substantially dorsiflexed nor plantarflexed). The spring is strong enough to overcome the internal resistance of the heel plate component but weak compared to the ground reaction force generated by heel strike. A pinion connected to the heel plate component can rotate in one direction freely (that is, the pinion free wheels) which enables the heel plate component to return to its neutral position with only the weak spring to drive it. As the heel plate component touches the ground during heel strike, the wearer's weight is transferred to the prosthetic particularly at the heel which is momentarily the only part of the prosthetic that is touching the ground. The pinion now wants to climb the rack in the direction opposite the freewheeling direction. Because the pinion is coupled to an axle, the pinion and axle turn as weight on the prosthetic foot forces the heel plate component into dorsiflexion. The axle, in turn, is coupled to a collector which is prevented from back turning by a pawl. The collector winds up a length of flexible tensioning component which energizes (e.g., stretches) the energy storage component.

During the flat foot stride of the gate, the pawl prevents the collector from unwinding. As the wearer moves into toe-off, the forefoot plate component moves into dorsiflexion compared to its neutral position. When the forefoot plate component rotates to, for example, 10 degrees, a boss pushes the pawl out of contact with the collector allowing the energy storage component to release its stored energy. The heel plate component is already in neutral position due to the weak spring and cannot be forced into plantarflexion by stops that limit its plantarflexion rotation. Thus, the only way to release the stored energy is to force the forefoot plate component into plantarflexion. The forefoot plate component is already dorsiflexed by at least 10 degrees, so the stored energy springs the forefoot plate component back towards neutral creating a forward propulsion and a slight lifting force that raises the prosthetic foot off the ground. A weak spring and/or stops connected to the forefoot plate component bring the forefoot plate component back to neutral position once the energy released from the collector is consumed as propulsion allowing the prosthetic foot to hover above the ground substantially parallel to the ground in the swing phase.

Energy may be stored in the device to be released during toe-off by other aspects of the invention. As a non-limiting example, an arch may be incorporated into the forefoot plate component(s) such that the arch elastically deforms when weight is applied to the forefoot plate component(s) like how the arch of a foot deforms during walking.

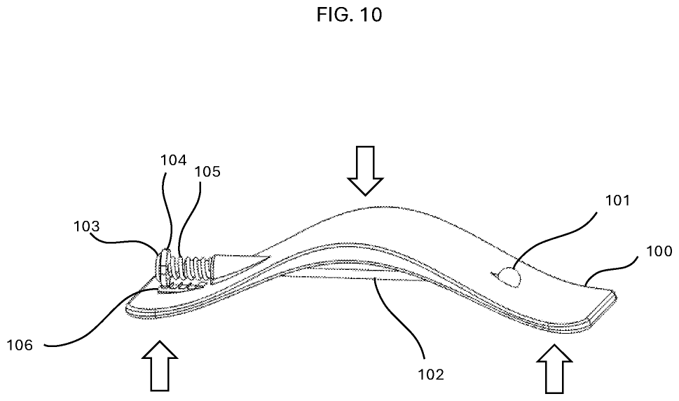

FIG. 10 is an orthogonal view of embodiment of an energy storage assembly comprising an arch plate (100), a ratcheting stop (106), and an energy storage element (105). The arch plate (100) can be made of carbon fiber, a metallic leaf spring, an elastomer, or the like. When a force is applied to the bottom ends and middle of the arch plate (as indicated by the block arrows), the arch plate flattens in shape. In aspects, the arch plate can be connected to a forefoot plate component wherein the downwards force is from the weight of the wearer and the upwards forces are from the ground reaction force at the ends of the arch plate. Alternatively, the arch plate could be incorporated into a heel plate component.

When the arch plate flattens, a rod (102) which is constrained by a ball (101) at one end, will draw the disc (103) at its second end towards the right (as shown in FIG. 10). This pushes a capture plate (104) into a compression spring (105). The capture plate is allowed to move to the right in FIG. 10 but is prevented from moving to the left by the ratcheting stop (106).

In this manner, work that is done to deflect (flatten) the arch plate is captured in the compression spring. When the forces flattening the arch plate are released, the arch plate springs back to its equilibrium state and the disc end of the rod (103) can move to the left unhindered. The ratcheting stop or prevents the compression spring from unloading even when the forces to flatten the arch plate have been removed.

When the ratcheting stop is rotated counterclockwise, the capture plate (104) is pushed to the left by the compression spring as it releases its stored energy by expanding in length. A line, rod, linkage, or the like may be connected to the capture plate to transfer the motion to another part of the overall assembly to which the energy storage assembly is connected. Likewise, the rotation of the ratcheting stop may be achieved by connecting it via a line, rod, linkage, or the like, to a different part of the overall assembly. For example, a line can be connected to a hinge such that when the hinge is flexed, the line pulls the ratcheting stop to release it. The capture plate can be connected via a linkage to the forefoot plate component of the overall assembly so that when energy is released the linkage pushes the forefoot plate component into the ground.

In aspects, the energy storage element can be a compression spring, a rubber compression spring, an elastomer, a tension spring, a torsional spring, flywheel, combinations thereof, or the like.

In the embodiments above, the heel and forefoot plate components are connected to the pylon at an axis of rotation. In aspects, a heel plate component and/or the forefoot plate component(s) are connected to a pylon by means of a multi-bar linkage. The linkage can be configured such that the bottom surface of the heel and/or forefoot plate components remains substantially parallel to the ground as the heel and/or forefoot plate components moves in dorsiflexion or plantarflexion.

An important consideration is that the heel plate component and the forefoot plate component return to a neutral position after heel lift off and toe-off. This neutral position is the position between dorsiflexion and plantarflexion when the foot is more or less parallel to the ground when the ankle is more or less perpendicular to the ground. In the neutral position, the toe or leading edge of the forefoot plate component skims above the ground as the leg swings forward. The toe or leading edge should be high enough above the ground such that it does not catch on irregular terrain causing the wearer to stumble or trip. Likewise, the heel plate component should return to neutral between heel lift off and heel strike so that it is in position to absorb the deceleration of heel strike.

A spring, weight, stops, or the like can be used to bring the foot plate components back to neutral when forces are not being applied to them (e.g., ground reaction forces or energy transfer forces from the energy storage and transfer mechanism).

When descending stairs or terrain it is sometimes preferable to touch down with the front of the foot rather than the heel first. Instead of heel strike being the first contact with the ground in a walking or running gait, a toe-touch may be employed in non-walking scenarios. In aspects, the ability to automatically or purposely switch from heel strike absorption to toe-touch absorption would be beneficial for some occasions. If the change from heel strike to toe-touch is made manually, the control mechanism for making the change is preferably at or below the waist for ease of use or at least towards the top of the pylon of the BK prosthetic.

In aspects, components or elements of the prosthetic device may be composed of a flexible composite (for example, carbon fiber composite). Composites are typically very strong and light weight which makes them desirable for prosthetic devices. It may be desirable to have some components have an elastic characteristic, for example, the toe region of the forefoot plate component. A flexible component is able to mimic the motion and reaction of certain parts of the foot anatomy (e.g., the flexing of the toes, the arch of the foot) without the need for hinges, movement, rotations between different elements. Friction, wear and tear, weight, and complexity are reduced which is desirable.

In aspects, it is desirable to absorb and transfer energy from one sub-assembly to another within the device. Non-compliant members are desirable to transfer forces efficiently. By analogy, a stiff crowbar is more effective at removing nails than a pliable crowbar.

The present invention can be optimized by using both compliant (flexible) and non-compliant (stiff) elements. A flexible carbon fiber composite arch in the forefoot plate component can be tuned provide a modicum of elastic recovery to accommodate uneven terrain. Beyond a certain applied force, the carbon fiber composite can be configured to “bottom out” (i.e., not be allowed to flex further) so that excess force can be transferred efficiently into energy absorbing and transferring mechanisms.

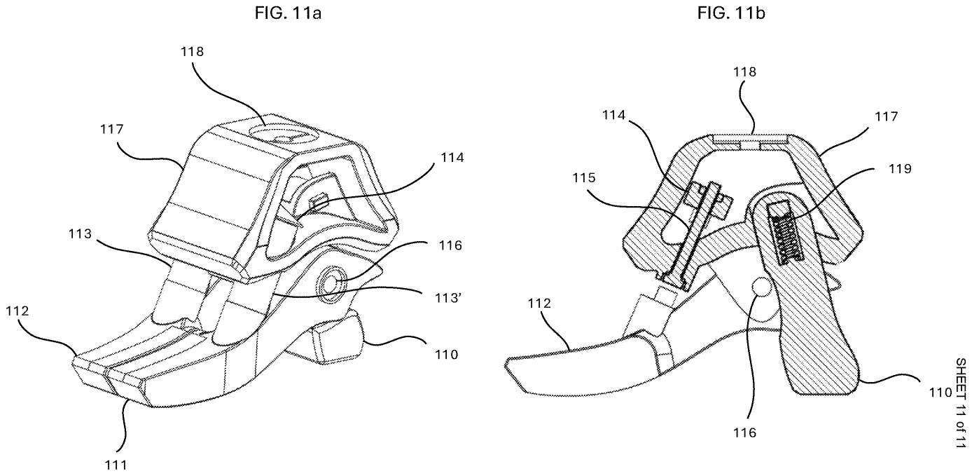

FIG. 11a and FIG. 11b show another embodiment of the present invention. FIG. 11a is an orthogonal view of a prosthetic foot comprising a heel plate component (110), a medial forefoot plate component (111), a lateral forefoot plate component (112), and a pylon receiver (117). The medial and lateral forefoot plate components are independently connected to the pylon receiver at a rotation axis (116). Polyurethane compression springs (113), (113′) are positioned between the upper surfaces of the medial and lateral forefoot plate components and bridge (114) that is connected to the pylon receiver. The compression springs (113), (113′) absorb the energy created by rotating the medial forefoot plate component (111) and the lateral forefoot plate component (112) clockwise, respectively. In aspects, the compression springs have similar spring constants so that the forces required to rotate the medial and lateral forefoot plate components are balanced. Alternatively, the compression springs may be selected with different characteristics to purposely impart more resistance to rotation for one forefoot plate component versus the other.

FIG. 11b is a cross sectional side view of the embodiment shown in FIG. 11a. (The compression springs (113) and (113′) are not illustrated in FIG. 11b.) A screw (115) is used to hold the bridge (114) to the pylon receiver (117). Tightening the screw draws the bridge closer to the tops of the compression springs which generates an adjustable preload. The heel plate component (110) slides within a channel of the pylon receiver. When weight is applied to the heel plate component, a spring (119) positioned between the heel plate component and the pylon receiver is compressed. The spring absorbs some of the heel strike force mimicking the natural action of the foot. A pylon can be attached to the pylon receiver at the receptacle (118).

The embodiment shown in FIG. 11a comprises two sets of energy storage elements. The first set of energy storage elements are the polyurethane spring (113, 113′) which store energy when the forefoot plate components are rotated into dorsiflexion. The second set of energy storage elements is the compression spring (119) which stores energy during heel strike. As illustrated in FIG. 11a and FIG. 11b, there are two independent adjustment mechanisms to adjust the preload on the two sets of energy storage elements. Adjusting the bridge (114) adjusts the preload on the two polyurethane springs. The spring (119) is mounted to the pylon receiver with a bolt that can be tightened to adjust the preload on the spring.

In addition, in aspects, the prosthetic can further comprise adjustable stops to set range of motion (ROM) of the various foot plate components. Using both the ROM stops, the adjustment mechanisms to set preloads, and the ability to exchange the energy storage elements with alternate energy storage elements with a different spring constant, the force distribution versus rotation (or translation) of the foot plate components can be programmed. As a non-limiting example, a hard ROM plantarflexion stop set at 10 degrees of flexion on the forefoot plate component(s) would prevent top drop. An energy storage element comprised of a 70 Shore A polyurethane compression spring with a length of approximately 1″ and a cross sectional area of about 0.25 sq in would store roughly 50 lbs. of toe-off force at 20 degrees of flexion with a preload compression on the compression spring of about ⅛″. Swapping the spring to a harder durometer polyurethane, a shorter length, a larger cross sectional area, or increased preload compression would increase the toe-off force at the same amount of flexion.

Further refinement of the force distribution provided by the energy storage elements can be achieved by changing the axial rotation of the various foot plate components with respect to the pylon receiver. For example, instead of a cylindrical axis (116) as shown in FIG. 11a, the forefoot plate components could be connected to the pylon receiver with a variable radiused track whereby the moment arm of the torque generated by compressing the energy storage element as the forefoot plate components rotate into dorsiflexion would vary with the amount of rotation. Similarly, cams, bicentric gears, elliptical guides, four bar linkages and the like that connect the (for example) forefoot plate components to the pylon receiver can be used to affect a non-uniform moment arm of the torque applied to the energy storage elements with the amount of rotation of the foot plate component(s).

Further adjustment of the energy storage during the gait cycles can be achieved by, for example, adjusting the stiffness of the various foot plate components. FIG. 7b illustrates a method of altering the stiffness of the forefoot plate component by hingedly attaching an anterior section to a posterior section. In addition to altering the connection elasticity via a set screw, a ratcheting dial can be used to allow the wearer to quickly change the elasticity of the hinge element. Using two or more materials (e.g. a carbon fiber anterior forefoot plate component and a nylon posterior forefoot plate component) could also be a means to change the stiffness of the various foot plate components. When two or more materials are used they may be mechanically connected or, in aspects, two or more different materials can be 3D printed as a unitary component with different properties along its geometry.

In aspects, hard elements (e.g., nylon) can be encapsulated in a softer matrix (e.g., TPU) to create a foot plate component that is able to rotate and/or translate without traditional mechanical connects (for example, bearings or slides). The hard elements would act like the bones of the foot and the soft matrix would act like the tendons and muscles that connect the bones. Adjustable tensioning or compression mechanisms can be used to squeeze the soft matrix various amounts or over varying locations to change the overall flexibility of the composite foot plate component.

As shown in FIG. 11a the pylon receiver has a receptacle (118) to accept the pylon of that is connected to the user's socket. In aspects, the socket and the prosthetic of the current invention is made substantially continuously via 3D printing. By using multi-material 3D printers, the socket that is worn of the user's residual limb could be made from a soft, compliant material (e.g., TPU) while the hard, stiff elements such as the foot plate components could be made from a hard material (e.g., nylon). For areas where exceptionally strong materials are needed, a pocket or pockets can be strategically positioned so that carbon fiber composite plates or pieces can be inserted after 3D printing. Such a combined prosthetic and socket could be made without any metal pieces (or a minimum of metal pieces such as, for example, less than 1 ounce of metal pieces) to reduce the weight of the article.

In aspects the forefoot plate component and heel plate component are comprised of many sub-units that are linked together with an elastic component such as a TPU strip, an extension spring, a compression spring, or the like. Changing the anchor point(s) of the elastic component can be used to change the effective spring constant of the elastic component, the direction of the force vector applied by the elastic component, or both. By dividing and connecting the foot plate components with elastic components, the flexibility and adaptability of the human foot can be better mimicked. Swapping out elastic components with different durometers (for example) can be a means to define how the foot plate component behaves. Likewise, adding or subtracting elastic components could also be used to change the flexibility of the foot plate component. Screws, set screws, bolts, clamps, pins, combinations thereof, and the like could be used to secure the elastic components to the foot plate components which could also have the effect of changing the effective flexibility. Such a system of interconnected sub-units would offer a high degree of customizability. It is anticipated that some of the settings would be changed in a prosthetics workshop (e.g., once and done) whereas other settings could be changed by the wearer to adapt to immediate needs (e.g., going up a staircase).

In aspects, motors and/or sensors can be incorporated to make the prosthetic of the present invention semi-automatic or fully automatic. An accelerometer could detect the moment of heel lift off and trigger a clutch to release the energy stored at heel strike to the forefoot plate component to propel the wearer at toe-off. A force sensor could detect the impact force at heel strike and adjust the tension in a bowline arch to stiffen up the foot plate component arch. Electric motors can be used to drive linear actuators or turn shafts. Alternatively, an electric pump can be used to pump hydraulic fluid to actuate various movable elements.

In embodiments, the present invention comprises a forefoot plate component and a heel plate component, wherein the forefoot plate component comprises at least one lever that articulates about one or more axes, and a heel plate component that comprises at least one lever that articulates about one or more axes, and each of the levers attached to a tensioning system on one end of the lever, and the other end of the lever makes contact with the ground, directly or indirectly, during use by the wearer.

In embodiments, the tensioning system comprises a cable or lace, an optional tensioning element, such as a spring or elastic member, and a tension adjustment mechanism, such as a ratcheting dial or motor. When the ratcheting dial is turned, the tension in the cables or lace increases, increasing the downward force to either or both of the forefoot or heel plate components. A shank representing the tibia is attached to the forefoot and heel plate components, and houses the tensioning element(s), cables/lace, and tension adjuster mechanism. The effect of the devices is that when the wearer strikes the ground with the heel plate (the heel), then the lever pulls on the tensioning system, thereby absorbing shock, and possibly canting the tibial shank forward during gait. Then the forefoot plate component lever is tensioned and absorbs shock through stance phase and provides a push-off force until the leg is lifted and the patient returns to swing phase.

In other aspects, the foot portion, or the interface of the foot portion comprises shock-absorbing materials, such as rubbers, TPUs, padding or gels. In aspects, the device can integrate directly or indirectly with a socket with beams, pylons, brackets, liners and the like.

In aspects, one or more of the forefoot or heel plates is comprised of an energy-storing material, such as a rubber, TPU, or carbon fiber element, to store and release energy during gait. In aspects, the device can be configured to absorb energy on impact and return energy upon plantar flexion/toe-off, to provide acceleration during gait.

The components in the device described herein may be a continuous or semi-continuous material with different mechanical properties where certain parts of the material bend or articulate differently than other parts of this semi-continuous material, wherein certain properties transition to materials with different properties. For example, nylon may transition to TPU for greater flexibility, and then transition back into nylon, to better represent a joint in a toe or transition from metatarsal to tarsal bone.

The components may further alternatively use hybrid materials, for example, carbon fiber or fiberglass, integrated with nylon to achieve ease of manufacturing with nylon, or pliability and/or improved strength, and to get the springiness of carbon fiber. This would also enable a higher degree of modularity where standardized carbon fiber components may be used with more bespoke and or a patient specific nylon component.

In aspects, the foot and ankle system may have a network of elastomeric components that can exert a multitude of forces. For example, a forefoot component may have the ability to both plantarflex and dorsiflex, like the human body. This can be controlled by many of the various force generators, including those mechanized. This concept can be applied to the knee (quads vs hamstrings) and elbow, back/spine, shoulder and wrist, for example.

In aspects, while the device is described as a prosthetic foot, similar designs and mechanisms may be applied in upper limb prostheses, such as a prosthetic hand or arm.

In aspects, the device described herein is a multi-axis rotation control foot prosthesis that can control forces around the joint of a prosthetic ankle, as well as be customized to the user's needs in various aspects. It can be adjusted by the user, manufacturer or clinician through multiple features including: 1) a tensioning or compression apparatus where the user can adjust the magnitude and direction of corrective force based their activity or indication, 2) optionally, a mechanism for changing the direction/orientation of corrective force or forces through a series of anchors, anchor points, channels, slots, or other positioning or connecting elements, and 3) optionally, a method to custom-fabricate the shape or form the device to the user's anatomy to optimize performance of the device and the user's gait.

Herein, ankle refers to the ankle joint of a prosthetic foot, or a joint that allows rotation between a forefoot and heel portion of the device, and the upper portion of the device. The joint is also referred to as a hinge. In some embodiments, the foot prosthesis is designed to limit motion in a specific direction, for example to prevent over rotation and counter rotation of the foot; this is accomplished by applying different amounts of tension or compression around different regions of the joint, and between some part of the foot or footwear and a part of the joint, the proximal part of the joint, or a part of the prosthesis situated above the joint. Tension is applied to manipulate the position of the foot relative to the below-knee socket. The applied tension can be fixed, adjustable, or dynamically adjustable in that tension increases with increasing motion between the prosthetic foot and socket. In other embodiments, the same directional forces may be applied to augment motion of the device in a desired direction. Through this mechanism, the device allows mobility within certain axes while limiting it in others to maximize the freedom of the joint while providing only the necessary directional stability.

Embodiments may be customized to optimally address the user's needs. This includes features such custom fabrication from a 3D-scan or measurements, custom fit through modification by a certified professional (e.g. thermoforming), adjusting the amount of force applied in a given direction using an adjustment mechanism connected to a tensioning element (e.g. containing elastic bands) or compression element (e.g. containing springs), or changing of the direction in which force is applied, including the axis about which a moment is generated. Tensioning or compression elements may be in the form of bands, sheets or lines and integrated within or external to the brace or sock. The size and geometry of these elements may be tailored to yield specific mechanical properties. Inelastic and elastic components may be combined in patterns or arrangements to control tension in areas where it is desired. In aspects, the device is provided as a modular kit, wherein components can be combined to create an optimized prosthesis for the patient.

### Mechanism for Adjusting the Magnitude of Force

The tensioning apparatus on the device allows for multiple aspects of customization for adjustment by the user, manufacturer or a certified professional (e.g. a doctor or physical therapist) for desired function, whether limitation of movement, augmentation of movement for rehabilitation, or performance enhancement. Among others, a novel aspect of the device relates to the ability to dynamically adjust the amount of tension in one or more directions around the joint, described herein as a multi-axis rotation control device. The term multi-axis means that tension can be generated across at least one axis. The amount of force generated in a given direction may be controlled dynamically or statically via energy storage elements in compression or tension. Energy storage elements are described as any element or component that stores mechanical energy including, for example, elastomeric bands, webs or other geometries, springs, pneumatic elements, electromagnetic elements, pneumatics, or any component that may act as a mechanical energy storage element that may be adjusted to hold variable forces. In aspects, the force within the element will be set by the user at a certain position, and will change throughout a range of motion.

For example, a series of bands on the lateral side of the device may be placed under tension to prevent inversion of the joint. In the same way, a compressive element (e.g. a spring) placed on the medial side of the joint may also limit or prevent inversion of the ankle to address the same indication. By adjusting the magnitude of force within the energy storage element, the device can replicate that natural properties and forces provided by organic foot-ankle complex tendons and muscles. In aspects, the invention can produce eccentric plantarflexion, for example, what would be required to go downstairs.

The user can adjust the magnitude of force within the tensioning or compression element using an adjustment mechanism. An adjustment mechanism allows the user or wearer of the device to increase, decrease or remove the amount of force stored within the tension or compression element and therefore the forces generated around the hinge/ankle joint. In aspects, the adjustment mechanism may comprise a dial, lever, ratchet and pawl system, pulley, electric motor, screw, etc. to modify and control dial in the amount of force supplied. The amount of force may be recommended by a prescriber or other professional and indicated on the device. The range of forces able to be applied by the mechanism may be prescribed by a professional, e.g. forces of 5-10 pounds may be achieved in the element through a construction or inclusion of certain components by the manufacturer or professional to support a gait pattern based on the user's characteristics. Other patients may benefit from less than 5 pounds or more than 10 pounds of tension. In other words, the device may incorporate tensioning elements of varying storage modulus, size, cross section, material or mechanical properties in general. By example, a tensioning element that may be adjusted with forces between 0-1 pound. to assist in device dorsiflexion may be used for weeks one and two of a rehabilitation regiment. The element may be replaced as rehabilitation progresses with bands of higher storage modulus, e.g. one that can hold up to 5 pounds of tensile force to aid in muscle strengthening.

In aspects, the ankle joint is configured to rotate around one to three axes of rotation. In some embodiments, the ankle joint is a ball and socket joint. The inclusion of an ankle joint in the prosthetic foot allows controlled inversion, eversion, dorsiflexion, and plantarflexion of the prosthetic foot in combination or independently from the forefoot plate component and heel plate component.

In aspects, the user may be able to rapidly and conveniently adjust the device during activity. For example, the user may engage tension in the device while walking, increase tension further while walking uphill, and disengage tension while seated within a span of seconds. The tensioning apparatus may also have preset values or ranges. The tensioning apparatus may be adjusted manually or automatically via a system of at least one sensor and motor or actuator. The adjustment mechanism may require a specific tool or tools to control force generation. For example, a special screw head may be required in changing tension to limit who has access to adjustments. This tool could be only available to the patient's clinician, parent, or guardian. The multiple tools could help to identify the level of force created. Different tools could apply tension with different maximum ranges to provide better control and force indication. Differing tools could be further used to identify which direction force is being applied. For example, with independent heel vs toe-off control, one tool would be used to adjust the heel and the other adjusts the toes.

The adjustment mechanism may provide a range of mechanical advantage depending on the design selected for the specific use case. For example, an adjustment mechanism providing a mechanical advantage of 2:1 may be used in conjunction with tensioning elements of a lower spring constant and for applications in which a lower amount of force is required to correct the ankle. In other examples, an adjustment mechanism with a 12:1 mechanical advantage may be selected in conjunction with higher durometer elastomers or for a high-torque application of the tensioning device, such as stabilizing the ankle during activity. A range of mechanical advantage from 2:1 to 24:1, by example, may be achieved in a dial-based adjustment mechanism. This mechanical advantage may be achieved in a compact design with a gear system which may include planetary gear systems, worm gears, or cycloidal gears and combinations thereof.

Alternatively, pulley systems incorporated into the device may provide a mechanical advantage in the adjustment mechanism. Alternatively, increasing or decreasing the lever arm of the tension adjustment device will provide different levels of achievable force.

The degree of force generated or stored within the tensioning or compressive element may be modified based on a user-controlled dial. This allows the user to change the force from zero to maximum based on activity. The degree of unloading may be modified by interchangeable elements, e.g. springs or bands of different spring constants. In addition to being able to adjust tensile force from zero to maximum continuously to achieve any force within the range, the device may allow for tensioning to specific discrete forces (e.g. in 0.1 lb., 0.2 lb., 0.3lb., 0.4 lb., 0.5 lb., 0.6 lb., 0.7 lb., 0.8 lb., 0.9 lb., 1.0 lb., 1.1 lb. increments), or may simply toggle between on and off (e.g. minimum and maximum force). The force may be adjusted in any number of increments including on or off, stepwise between a minimum and maximum force, or continuously on a gradient.

The tensioning elements may be constructed of different materials to provide different properties based on the user's need. For example, a tensioning element consisting of an elastomeric element of certain viscoelastic properties may better mimic the feel of natural muscle, tendon, and ligament complexes, while a more rigid or fully rigid tensioning element may completely lock the foot in one or more directions or prevent movement beyond a specific range of motion.

The tensioning elements may contain a system of springs in parallel or series, in which springs can be added or removed to adjust tension. For example, a tensioning element consisting of one active spring, may have a second or third spring element engaged in parallel to increase the total force of the tensioning system. By adjusting the control mechanism, the user is adding or removing springs from the system.

Configurations of embodiments can also be adopted for an ankle foot orthosis, rather than a prosthesis. The energy storage and transfer mechanisms described above can be used in an ankle foot orthosis (AFO) by the tensioning of a forefoot plate component (or one or more foot plate component components) to create a plantarflexion moment in the forefoot. Alternatively, the heel plate component shock absorbing component can be configured to provide a heel push-off force. Whereas a prothesis has a pylon to connect with the wearer's limb, an AFO variant would have one or more side struts to connect the foot components to the wearer's calf. This type of brace can unload or offload the ankle joint which may be helpful for a number of indications, for example ankle arthritis.

Other configurations can use sensors and/or actuators which may be hydraulic, electric, or using electronically-controlled hydraulics. These powered system systems can involve tensioning either forefoot plate component components, heel plate component components, or both. One variant involves a powered dorsi-assist system to lift the toe through a period of gait cycle, usually to avoid toe catch, and then can produce a passive or active forefoot push off for force. Controls of a powered or active system can be predefined or may use an artificial intelligence-based processor to make control decisions in real or near-real time by interpreting information or terrain and/or the environment. Artificial intelligence can be used in other ways, for example, generative design to meet design requirements for a given patient considering their health and/or physical and physiological needs. Such systems can be active, passive, or a hybrid system. The powered variant devices described herein can be connected neurally to the user, allowing the patient to control the device with their brain or movement of other limbs or body parts, and with or without the help of AI.

The prosthetic foot or orthotic AFO can be integrated with other orthotic and prosthetic components and controls for these devices can be coupled to provide a synergistic compound action, or to simplify controls by having fewer individual tension controllers. For example, the ankle component can be integrated with a prosthetic assistive knee joint using Icarus technology that uses elastomers drawn across the joint and the tension can be adjusted to change the assistive force, and the adjustment for the knee can be independent of the ankle or connected to the ankle.

### Mechanism for Adjusting the Direction of Force

The direction of force may be modified statically or dynamically by the user, manufacturer, or a certified individual to address a specific need. An anchor may be connected at anchor points between the two elements: one at the proximal cuff and one or more at the distal portion, which yield a net force generated around the AP axis to prevent inversion. Multiple anchor points may be distributed along the proximal or distal cuff to provide greater stability and force distribution. This distribution may be created as a function of the geometry of the tensioning or compression element, which yields a net force vector controlled through one or more adjustment mechanism that optimally reduces inversion or eversion. For example, the shape and position of an elastomeric band, sheet or web between the proximal and distal portions will determine the force magnitude, direction, and overall distribution depending on the path it generates between the proximal portion and distal portion. The cables or tensioning elements may be routed through rigid or flexible material, or along the side or surface of the brace or sock to generate the desired force profile across the proximal and distal portions of the device. For example, the device meant to prevent foot drop will have cables and/or tensioning elements routed near the front of the foot above the ankle that have anchor points in the distal portion of the device, in a position that is forward from the position where the cables or tensioning elements connect in the proximal portion of the device. In other aspects, the device may mimic dorsiflexion by the tensioning system being anchored to the front part of the foot or footwear and generate an adjustable torque along the ML axis. The user can increase tension to provide additional support for dorsiflexion.

The tensioning or compression element may be anchored between the proximal and distal portions to limit or support abduction or adduction.

As a multi-axis, rotation control device, the tensioning or compression elements may be anchored in a way that supports multiple directions of movement in the face of multi-directional deficiency. A dynamically adjustable device allows each user's specific need to be addressed optimally in a single device.

In the proximal portion, the tensioning elements may all be anchored at one point directly or indirectly to the adjustment mechanism. However, the direction of force and relative tension in each of the one or more tensioning elements may be modified by channels, grooves, guides or other structures on the proximal cuff, herein described generally as channels.

The anchor points or lace paths may be determined to optimize function through algorithms, artificial intelligence, machine learning, and general software-automated design of the fabricated device. The anchor points or connection of the tensioning element may be modified and placed at any point or angle of the proximal or distal portion of the device. There may be unlimited orientations and positions across the surface of the device. Alternatively, the anchor points may be pre-set to limit options for force direction.

### Adjustment Mechanism

In order to control forces and rotation around one or more axes, the device may contain multiple tensioning or compression elements to address the user's need. In aspects, one or more tensioning or compression elements may be controlled by a single adjustment mechanism. The tensioning elements can be modular and added as needed based on the user's condition.

Additionally, the device can contain one or more adjustment mechanisms of the same or varying design (as non-limiting examples, e.g., rotary dials, levers, ratchet and pawl mechanisms, screws, or motors) that change the magnitude of force within the tensioning or compression element(s) around one or more axes.

The adjustment mechanisms may also change the direction of force by moving anchor points, changing tensioning or compression element(s) geometry, or changing orientation of the tensioning or compression element(s).

The user may be able to remove the adjustment mechanism, or may be able to move the adjustment mechanism from one point on the device to another. Multiple adjustment mechanisms may be incorporated on the device to modify force in multiple directions. The adjustment mechanism may be in the form of a slot with a corresponding external component, e.g. a key that can be inserted at multiple locations of the device to adjust forces in different orientations, engage or disengage different tensioning or compression elements, or change the direction of force.

### Motors and Sensors

The tensioning or compressive elements may increase or decrease force across, between, or within the proximal and distal portions through adjustment by a motor. The motor may be controlled by an electronic interface connected directly or indirectly to the device. The interface may be an app or software on the user's mobile device, smart watch, or other computer/smart device. In aspects, the device may comprise one or more processors, one or more motors, one or more controllers, one or more sensors, one or more antennas, as well as software to control, instruct, process, command, implement adjustment of the tensioning element(s) of the device. The motor or elements may control or change force within the tensioning or compression element(s) automatically based on input from sensors located on the device or elsewhere on the user. For example, EMG sensors located in the below knee socket and/or accelerometers may detect stages of gait to optimize positioning of the foot and resulting gait throughout a range of motion. In another example, sensors may trigger the energy storage units to engage while stepping to support device plantarflexion at one stage of gait and support device dorsiflexion at another. In other aspects, the tensioning element can be manually adjusted using, for example, a smartphone, smart wearable technology, computer, external processor, or an electronic control mechanism on or attached to the device. These adjustments can be made in real-time or substantially real-time as understood by one of skill in the art; they can be made by the user or a treating practitioner; and/or they can be made while the user is wearing the device. A combination of both automatic and manual adjustment is also contemplated.

### Method of Making

The various embodiments of the present disclosure may use traditional manufacturing processes for prostheses and/or 3D printing/additive manufacturing to produce the components and/or the overall device.

The fabrication technique allows low-cost, custom devices. It also enables manufacturing of intricate parts containing internal channels and features that could not be feasibly or affordably produced with injection molding, machining, or other traditional methods of manufacturing orthotic devices. 3D printing enables efficient production of lightweight, yet durable materials such as thermoplastics. The result is a highly effective, lightweight, customized, and cost-effective device that can be manufactured at scale.

The prosthetic and components thereof can also be personalized to suit an individual's preference or mechanical needs. For example, the prosthetic can be made in a similar shape, weight, and/or balance to another part of the body. The prosthetic may have more than one axis of rotation as shown, for example in FIG. 8, which approximate one or more joints in the anatomical foot and ankle and leg, or different synthetic joint. For example, the forefoot component may have two toes for one patient that are tensioned, and another may have four. The design of devices can be highly automated and designed partially or fully by artificial intelligence or machine learning.

Carbon fiber elements may be included where additional strength or the need for particular elastic properties are required.

In embodiments, the device can mimic aspects of the anatomy of the human leg and has potential to feel more natural, weigh less, and be readily tailor able while the user is wearing the device. In aspects, it allows for active plantarflexion that may or may not be controlled by a microprocessor. In aspects, the device can articulate in a myriad of ways to allow the foot to conform to uneven terrain.

In aspects, the hinge or ankle joint between the forefoot plate component(s), the heel plate component(s), and the shaft component is a ball joint. In aspects, a tension or compression element surrounds the ball joint and is anchored to the upper portion and the lower portion. The joint can freely rotate, but resistance is provided by the tensioning or compression element, which can be tightened or loosened with an adjustment mechanism to limit range of motion of the joint. In other aspects, the device comprises a drop-lock mechanism, swing phase lock mechanism, or other similar mechanism wherein articulation or movement of one portion of the device (or another part of the user's body) causes the release of a latch, an engagement of the tensioning/compression element, or a disengaging of the tensioning or compression element to release or engage a component of the device. In other aspects, the device can incorporate a single or multi axis hinge.

### Other Embodiments and Aspects of the Invention

Embodiments of the current invention can be designed to incorporate novel articulating and dynamic-response mechanisms that store and return energy during gait in order to approximate the biomechanical function of the anatomical ankle-foot complex. In designs, the prosthetic foot is comprised of discrete heel, ankle, and forefoot or toe regions that rotate, translate or deform elastically under load. Considering the anatomy of the foot, which is comprised of a large number of articulating joints, components including the forefoot and heel foot may be comprised of more than one articulating member to provide additional flexibility, control, and degrees of freedom to more naturally mimic natural gait patterns. In aspects, when a user loads the prosthesis during stance phase, the deformation energy storage elements or components store mechanical energy that can subsequently be released during push-off to assist propulsion. Carbon-fiber composite leaf springs, segmented keels, and elastomeric compression members are commonly used for this purpose because they provide high strength-to-weight ratios and predictable elastic behavior under repeated loading cycles. Such energy-storing feet operate analogously to springs, converting kinetic energy from ground reaction forces into stored potential energy and then returning a portion of that energy during late stance and toe-off.

The heel portion of a prosthetic foot may include compressible or pivoting components configured to permit controlled plantarflexion immediately following heel strike. For example, the heel section may comprise an elastomeric wedge, curved composite beam, or hinged heel plate component that deflects under initial loading. This deformation absorbs impact forces and stores elastic energy while allowing the prosthesis to smoothly transition from heel strike to foot-flat. Mechanical testing has demonstrated that heel elements in prosthetic feet exhibit measurable stiffness characteristics and that increased heel stiffness typically correlates with greater resistance to deformation and altered energy storage behavior during the early stance phase. Such behavior can be tuned based on the design of the heel plate component, the incorporation of an adjustment mechanism to be controlled by the user, or the shape, cross sectional area, modulus, spring constant, or mesh design of an energy storage element that is coupled to the heel plate component.

The forefoot and toe regions of the prosthetic foot may include articulating toe segments or flexible forefoot keels that bend during terminal stance and pre-swing. Toe joints are particularly significant because they exhibit nonlinear stiffness properties that influence the rollover characteristics of the prosthetic foot and the kinetics of the ankle during push-off. In some designs, a segmented forefoot or toe hinge enables the prosthetic foot to mimic the metatarsophalangeal joint of the human foot, allowing controlled dorsiflexion of the toe portion under load. The resulting elastic deflection provides a spring-like effect that enhances forward propulsion and contributes to the perception of “springiness” during toe-off. Energy storage elements may be independently tensioned or compressed, and may have variable design or composition between the multiple regions or components of the forefoot portion.

Other embodiments further include an articulating ankle joint mechanism positioned between the pylon interface and the foot structure. The ankle joint may incorporate rotational pivots, elastomeric bushings, torsion springs, or leaf-spring structures configured to permit controlled dorsiflexion and plantarflexion. These ankle mechanisms may function in conjunction with the heel and forefoot components to distribute loads across the gait cycle while storing and releasing elastic energy. Variable-stiffness ankle systems, comprising torsional spring assemblies or compliant mechanisms allow the effective joint stiffness to change during walking, thereby improving gait efficiency and user comfort.

In certain embodiments, the prosthetic foot may incorporate adjustable mechanisms that allow a user or clinician to tune the mechanical response of the device. Such adjustability may include user-selectable stiffness categories, interchangeable spring elements, adjustable compression members, or dial-controlled mechanisms that vary the preload applied to elastic components. By adjusting these parameters, the user may modify the range of motion, energy storage characteristics, and effective stiffness of the heel, ankle, or forefoot segments. This tunability allows the prosthesis to accommodate differences in user weight, activity level, terrain, or footwear while maintaining appropriate rollover dynamics and energy return during gait.

Additionally, the prosthetic foot may include mechanisms for adjusting the allowable range of motion of one or more articulating joints. For example, adjustable stops, cam mechanisms, peg and slot systems, inelastic tensioning elements, or threaded limiters may be used to define maximum dorsiflexion or plantarflexion angles of the ankle, heel or toe joint. Similarly, adjustable damping or spring-preload systems may regulate the magnitude and rate of energy storage and release. Through such mechanisms, the prosthesis may be configured to provide a customized combination of compliance, stability, and propulsion tailored to the preferences and functional needs of the user.

In embodiments involving energy storage components in tension, the same function may often be achieved by positioning an energy storage component in compression on the opposite side of a plate or rigid component (e.g., a forefoot plate component or heel plate component).

The embodiments described herein have various non-limiting Aspects:

Aspect 1: A foot prosthetic comprised of at least one forefoot plate component that pivots about a first axis, a heel plate component that pivots about a second axis, and an energy storage and transfer mechanism, wherein the ground force reaction on the heel plate component generated during heel strike is stored and transferred into a forward propulsion force to the at least one forefoot plate component during toe-off.

Aspect 2: The foot prosthetic of Aspect 1, wherein the first axis and the second axis are co-linear.

Aspect 3: The foot prosthetic of Aspect 1, wherein the at least one forefoot plate component is pivotedly connected to a pylon along the first axis.

Aspect 5: The foot prosthetic of Aspect 1, wherein the energy storage and transfer mechanism comprises at least one flexible tensile component, at least one energy storage component, and at least one tension adjustment component.

Aspect 6: The foot prosthetic of Aspect 5, wherein the at least one flexible tensile component is in-line with the at least one energy storage component.

Aspect 7: The foot prosthetic of Aspect 1, wherein a one-way clutch connected to the heel plate component allows the heel plate component to load the energy storage and transfer mechanism during heel strike and prevents the energy storage and transfer mechanism from unloading the stored energy back to the heel plate component.

Aspect 8: The foot prosthetic of Aspect 1, wherein energy stored in the energy storage and transfer mechanism is not transferred to the at least one forefoot plate component until the forefoot plate component is inclined into dorsiflexion.

Aspect 9: The foot prosthetic of Aspect 8, wherein the inclination into dorsiflexion is greater than 10 degrees.

Aspect 10: The foot prosthetic of Aspect 5, wherein the energy storage component dissipates less than 20 percent of the stored energy after 0.1 second.

Aspect 11: The foot prosthetic of Aspect 10, wherein the energy storage component dissipates more than 50 percent of the stored energy after 1 second.

Aspect 12: The foot prosthetic of Aspect 1, wherein the at least one forefoot plate component comprises an arch with a natural resistance to flexing, at least one stiffening component functionally connected to the arch, wherein altering or adjusting the at least one stiffening component changes the flexibility of the arch.

Aspect 13: The foot prosthetic of Aspect 12, wherein the at least one forefoot plate component comprises a medial forefoot plate component that pivots about the first axis, and a lateral forefoot that pivots about the first axis, wherein the degree of rotation of the medial forefoot plate component may be different than the degree of rotation of the lateral forefoot plate component.

Aspect 14: A foot prosthetic comprised of at least one forefoot plate component that pivots about a first axis, a heel plate component glides along a track, and an energy storage and transfer mechanism, wherein the heel dampens the ground force reaction generated during heel strike is stored and transferred into a forward propulsion force to the at least one forefoot plate component during toe-off.

Aspect 15: The foot prosthetic of Aspect 14, wherein the at least one forefoot plate component is pivotedly connected to a pylon along the first axis.

Aspect 16: The foot prosthetic of Aspect 14, wherein the at least one heel plate component glides along a linear path, wherein the path is in a non-intersection direction to the first axis.

Aspect 17: The foot prosthetic of Aspect 14, comprising at least one force adjustment mechanism and at least one elastic component.

Aspect 18: The force adjustment mechanism of Aspect 17 comprising of at least one plate component that preloads the elastic component, wherein the plate component's position can be adjusted by the wearer to increase or decrease force on the elastic component.

Aspect 19: The plate component of Aspect 18, wherein the plate component's position can be adjusted by screwing a bolt that threads into the plate component.

Aspect 20: The foot prosthetic of Aspect 1, wherein the first axis and the second axis are the same.

Aspect 21: The foot prosthetic of Aspect 1, further comprising an ankle joint, wherein the ankle joint is capable of pivoting around one or more axes of rotation.

Aspect 22: A foot prosthetic comprised of at least one forefoot plate component that pivots about an axis, a heel plate component that pivots about an axis, and at least one energy storage component, wherein the energy storage component is compressed, elongated, or otherwise deformed to store energy upon rotation or translation of at least of the forefoot plate component or the heel plate component, and wherein the stored energy is released during the user's gait.

Aspect 23: The foot prosthetic according to Aspect 22, further comprising an adjustment mechanism, wherein the adjustment mechanism is capable of changing the amount of stored energy within the energy storage component, translating or rotating the forefoot plate component, the heel plate component, or both.

Aspect 24: The foot prosthetic according to Aspect 23, further comprising a flexible tensioning component, wherein the flexible tensioning component couples the adjustment mechanism to the energy storage component.

Aspect 25: The foot prosthetic according to Aspect 22, wherein at least one of the forefoot plate component, the heel plate component, the energy storage component, or other components of the foot prosthetic are modular and can be designed, selected, or interchanged to meet the specific needs of the user.

Aspect 26: The foot prosthetic according to Aspect 22, wherein the adjustment mechanism is a dial, a lever, a ratchet-pawl system, a switch, a digital interface, one or more buttons, or a pull tab.

Aspect 27: The foot prosthetic according to Aspect 22, wherein the energy storage component stores energy in compression, and wherein rotation or translation of the forefoot plate component, the heel plate component, or both during gait causes compression energy to be stored and released within the energy storage component.

Aspect 28: The foot prosthetic according to Aspect 22, further comprising a pivoting ankle joint, wherein the pivoting ankle joint is capable of storing and releasing energy during gait around one of more axes of the ankle joint.

Embodiments of the invention include a computer readable medium comprising one or more computer files comprising a set of computer-executable instructions for performing one or more of the calculations, steps, processes and operations described and/or depicted herein. In exemplary embodiments, the files may be stored contiguously or non-contiguously on the computer-readable medium. In aspects, the files or data may be sent directly or indirectly to the cloud or remote servers(s). Embodiments may include a computer program product comprising the computer files, either in the form of the computer-readable medium comprising the computer files and, optionally, made available to a consumer through packaging, or alternatively made available to a consumer through electronic distribution. As used in the context of this specification, a “computer-readable medium” is a non-transitory computer-readable medium and includes any kind of computer memory such as floppy disks, conventional hard disks, CD-ROM, Flash ROM, non-volatile ROM, electrically erasable programmable read-only memory (EEPROM), and RAM. In exemplary embodiments, the computer readable medium has a set of instructions stored thereon which, when executed by a processor, cause the processor to perform tasks, based on data stored in the electronic database or memory described herein. The processor may implement this process through any of the procedures discussed in this disclosure or through any equivalent procedure.

In other embodiments of the invention, files comprising the set of computer-executable instructions may be stored in computer-readable memory on a single computer or distributed across multiple computers or involve a network of remote servers hosted on the internet. In aspects, local, edge, or remote computing possibilities are used to store, manage, and process data. A skilled artisan will further appreciate, in light of this disclosure, how the invention can be implemented, in addition to software, using hardware or firmware. As such, as used herein, the operations of the invention can be implemented in a system comprising a combination of software, hardware, or firmware.

Embodiments of this disclosure include one or more computers or devices loaded with a set of the computer-executable instructions described herein. The computers or devices may be a general-purpose computer, a special-purpose computer, or other programmable data processing apparatus to produce a particular machine, such that the one or more computers or devices are instructed and configured to carry out the calculations, processes, steps, operations, algorithms, statistical methods, formulas, or computational routines of this disclosure. The computer or device performing the specified calculations, processes, steps, operations, algorithms, statistical methods, formulas, or computational routines of this disclosure may comprise at least one processing element such as a central processing unit (i.e., processor) and a form of computer-readable memory which may include random-access memory (RAM) or read-only memory (ROM). The computer-executable instructions can be embedded in computer hardware or stored in the computer-readable memory such that the computer or device may be directed to perform one or more of the calculations, steps, processes, and operations depicted and/or described herein.

Additional embodiments of this disclosure comprise a computer system for carrying out the computer-implemented method of this disclosure. The computer system may comprise a processor for executing the computer-executable instructions, one or more electronic databases containing the data or information described herein, an input/output interface or user interface, and a set of instructions (e.g., software) for carrying out the method. The computer system can include a stand-alone computer, such as a desktop computer, a portable computer, such as a tablet, laptop, PDA, or smartphone, or a set of computers connected through a network including a client-server configuration and one or more database servers. The network may use any suitable network protocol, including IP, UDP, or ICMP, and may be any suitable wired or wireless network including any local area network, wide area network, Internet network, telecommunications network, Wi-Fi enabled network, or Bluetooth enabled network. In one embodiment, the computer system comprises a central computer connected to the internet that has the computer-executable instructions stored in memory that is operably connected to an internal electronic database. The central computer may perform the computer-implemented method based on input and commands received from remote computers through the internet. The central computer may effectively serve as a server and the remote computers may serve as client computers such that the server-client relationship is established, and the client computers issue queries or receive output from the server over a network.

The input/output interfaces may include a graphical user interface (GUI) which may be used in conjunction with the computer-executable code and electronic databases. The graphical user interface may allow a user to perform these tasks through the use of text fields, check boxes, pull-downs, command buttons, and the like. A skilled artisan will appreciate how such graphical features may be implemented for performing the tasks of this disclosure. The user interface may optionally be accessible through a computer connected to the internet. In one embodiment, the user interface is accessible by typing in an internet address through an industry standard web browser and logging into a web page. The user interface may then be operated through a remote computer (client computer) accessing the web page and transmitting queries or receiving output from a server through a network connection.

The present invention has been described with reference to particular embodiments having various features. In light of the disclosure provided above, it will be apparent to those skilled in the art that various modifications and variations can be made in the practice of the present invention without departing from the scope or spirit of the invention. One skilled in the art will recognize that the disclosed features may be used singularly, in any combination, or omitted based on the requirements and specifications of a given application or design. When an embodiment refers to “comprising” certain features, it is to be understood that the embodiments can alternatively “consist of” or “consist essentially of” any one or more of the features. Other embodiments of the invention will be apparent to those skilled in the art from consideration of the specification and practice of the invention.

It is noted that where a range of values is provided in this specification, each value between the upper and lower limits of that range is also specifically disclosed. The upper and lower limits of these smaller ranges may independently be included or excluded in the range as well. The singular forms “a,” “an,” and “the” include plural referents unless the context clearly dictates otherwise. It is intended that the specification and examples be considered as exemplary in nature and that variations that do not depart from the essence of the invention fall within the scope of the invention. Further, all of the references cited in this disclosure are each individually incorporated by reference herein in their entireties and as such are intended to provide an efficient way of supplementing the enabling disclosure of this invention as well as provide background detailing the level of ordinary skill in the art.

As used herein, the term “about” refers to plus or minus 5 units (e.g., percentage) of the stated value.

Reference in the specification to “some embodiments”, “an embodiment”, “one embodiment” or “other embodiments” means that a particular feature, structure, or characteristic described in connection with the embodiments is included in at least some embodiments, but not necessarily all embodiments, of the inventions.

As used herein, the term “substantial” and “substantially” refers to what is easily recognizable to one of ordinary skill in the art.

It is to be understood that the phraseology and terminology employed herein is not to be construed as limiting and are for descriptive purpose only.

It is to be understood that while certain of the illustrations and figure may be close to the right scale, most of the illustrations and figures are not intended to be of the correct scale.

It is to be understood that the details set forth herein do not construe a limitation to an application of the invention.

Furthermore, it is to be understood that the invention can be carried out or practiced in various ways and that the invention can be implemented in embodiments other than the ones outlined in the description above.

1. A foot prosthetic comprising:
a forefoot plate component that pivots about a first axis;
a heel plate component that pivots about a second axis; and
an energy storage component;
wherein the energy storage component is compressed, elongated, or otherwise deformed, to store energy upon rotation or translation of the forefoot plate component, the heel plate component, or both, at a stage in a user's gait cycle, and wherein the stored energy is released at a later stage in the user's gait cycle.

2. The foot prosthetic according to claim 1, further comprising an adjustment mechanism, wherein the adjustment mechanism is capable of changing an amount of the stored energy within the energy storage component.

3. The foot prosthetic according to claim 2, further comprising a flexible tensioning component, wherein the flexible tensioning component couples the adjustment mechanism to the energy storage component.

4. The foot prosthetic according to claim 1, wherein the forefoot plate component, the heel plate component, and the energy storage component, are modular and designed, selected, chosen, or interchangeable, to meet one or more medical needs of the user of the foot prosthetic.

5. The foot prosthetic according to claim 2, wherein the adjustment mechanism comprises a dial, a lever, a ratchet-pawl system, a switch, a digital interface, one or more buttons, or a pull tab.

6. The foot prosthetic according to claim 1, wherein the first axis and the second axis are the same.

7. The foot prosthetic according to claim 1, wherein the first axis and the second axis are colinear.

8. The foot prosthetic according to claim 1, wherein the energy storage component stores energy in compression.

9. The foot prosthetic according to claim 1, further comprising a pivoting ankle joint, wherein the pivoting ankle joint is capable of storing and releasing energy during the user's gait cycle around one of more axes of the pivoting ankle joint.

10. A foot prosthetic comprising:
a forefoot plate component that pivots about a first axis;
a heel plate component that pivots about a second axis; and
an energy storage component;
wherein the energy storage component collects and stores energy during a heel strike stage in a user's gait cycle, and wherein at least some of the stored energy is transferred to the forefoot plate component and operative to propel the user during a toe-off stage of the user's gait cycle.

11. The foot prosthetic of claim 10, wherein the first axis and the second axis are the same.

12. The foot prosthetic of claim 10, wherein the first axis and the second axis are co-linear.

13. The foot prosthetic of claim 10, wherein the forefoot plate component is pivotably connected to a pylon along the first axis.

14. The foot prosthetic of claim 10, further comprising a flexible tensile component and a tension adjustment component.

15. The foot prosthetic of claim 14, wherein the flexible tensile component is in-line with the energy storage component.

16. The foot prosthetic of claim 10, further comprising a one-way clutch configured to store energy in the energy storage component from the heel plate component during the heel strike stage in the user's gait cycle and prevent unloading of the stored energy back to the heel plate component.

17. The foot prosthetic of claim 10, wherein energy stored in the energy storage component is transferred to the forefoot plate component when the forefoot plate component is inclined into a dorsiflexion position.

18. The foot prosthetic of claim 17, wherein the inclination into the dorsiflexion position is greater than about 10 degrees.

19. The foot prosthetic of claim 10, wherein the energy storage component dissipates less than about 20 percent of stored energy about 0.1 second after heel strike.

20. The foot prosthetic of claim 10, wherein the energy storage component dissipates more than about 50 percent of stored energy about 1 second after heel strike.

21. The foot prosthetic of claim 10, wherein the forefoot plate component comprises an arch resistance to flexing and a stiffening component connected to the arch, and wherein altering or adjusting the stiffening component alters a flexibility of the arch.

22. The foot prosthetic of claim 10, wherein the forefoot plate component comprises a medial forefoot plate component that pivots about the first axis, and a lateral forefoot plate component that pivots about the first axis, and wherein a degree of rotation of the medial forefoot plate component is different, equivalent, or substantially equivalent, compared to a degree of rotation of the lateral forefoot plate component.

23. A foot prosthetic comprising:
a forefoot plate component that pivots about a first axis;
a heel plate component that slides along a track; and
an energy storage component;
wherein the energy storage component collects and stores energy during a heel strike stage in a user's gait cycle, and wherein at least some of the stored energy is transferred to the forefoot plate component to propel the user during a toe-off stage of the user's gait cycle.

24. The foot prosthetic of claim 23, wherein the forefoot plate component is pivotably connected to a pylon along the first axis.

25. The foot prosthetic of claim 23, wherein the track upon which the heel plate component slides does not intersect the first axis.
